(19) United States
(12) Patent Application Publication
(10) Pub. No.: US 2019/0362654 A1
(43) Pub. Date: Nov. 28, 2019

(54) INNER LIMITING MEMBRANE PEELING MODEL AND USE THEREOF

(71) Applicant: National University Corporation Nagoya University, Aichi (JP)

(72) Inventors: Seiji Omata, Aichi (JP); Takeshi Hayakawa, Aichi (JP); Shinya Sakuma, Aichi (JP); Fumihito Arai, Aichi (JP)

(73) Assignee: National University Corporation Nagoya University, Aichi (JP)

(21) Appl. No.: 16/463,483

(22) PCT Filed: Nov. 16, 2017
(86) PCT No.: PCT/JP2017/041318

§ 371 Date: May 23, 2019

(30) Foreign Application Priority Data
Nov. 24, 2016 (JP) 2016-227731

(51) Int. Cl. G09B 23/30; A61F 9/00; A61F 9/007

(52) U.S. Cl. CPC G09B 23/30; A61M 2025/0042; A61F 9/007; A61F 9/0017

(57) ABSTRACT

The invention provides an inner limiting membrane peeling model that is used in procedure training in peeling of the inner limiting membrane. The model has: a simulated retina and a simulated inner limiting membrane formed on the simulated retina.

The simulated inner limiting membrane is formed of a hydrophilic polymer gel that is formed mainly of a water-soluble polymer. At least when used in the above procedure training, the model is disposed with the simulated inner limiting membrane immersed in water or an aqueous solution containing a predetermined solute.

### TECHNICAL FIELD

The present invention relates to an inner limiting membrane peeling model for ophthalmic use, the model being used, for instance, in procedure training of surgery for peeling an inner limiting membrane of the eyeball, and also relates to uses of the inner limiting membrane peeling model.

The present application claims the benefit of priority to Japanese Patent Application No. 2016-227731, filed on Nov. 24, 2016, which is incorporated by reference in its entirety.

### BACKGROUND ART

The outer wall of the human and mammalian eye is provided with various membrane tissues such as the sclera, the choroid and the retina, as viewed from outside the eyeball. Furthermore, a thin membrane tissue referred to as the inner limiting membrane (ILM) is present on the inward side of the retina (the vitreous side of the eyeball will be referred to herein as “inward/inner”; likewise hereinafter).

Damage or disorders in part of the retina may result, for instance, in reduction of visual acuity, distortion of the visual field and visual field loss, all of which may further progress to blindness. For instance, disorders in the macula (particularly in the foveal area) give rise to significant vision loss or central vision loss, which translates into a decrease in quality of life (QOL). Such disorders constitute therefore a major target for therapy in ophthalmology.

An example of such disorders (diseases) is the condition called macular hole, in which the macula is perforated. Treatment of the macular hole involves ordinarily vitreous surgery. Specifically, the posterior vitreous is peeled, the inner limiting membrane is peeled, and liquid/gas replacement is performed. Peeling of the inner limiting membrane has not been an essential treatment in the therapy of macular hole. Through peeling of the inner limiting membrane, however, the extensibility (flexibility) of the retina around the hole is increased and the hole becomes likelier to close (i.e. the effect of the treatment is more pronounced), and accordingly peeling of the inner limiting membrane has come to be performed frequently in recent years. Moreover, peeling of the inner limiting membrane has been reported to be excellent in dealing with, for instance, disorders in macular epithelia, and retinal vein occlusion, and thus inner limiting membrane peeling has earned a position as an important procedure in vitreous surgery.

Meanwhile, inner limiting membrane peeling is a surgical procedure requiring a particularly delicate technique, since the inner limiting membrane is extremely thin (the average membrane thickness of the inner limiting membrane of the human eyeball is about 3 μm), and the retina is present directly under the inner limiting membrane. Therefore, it is particularly important that prior training in peeling of the inner limiting membrane be recurrently carried out, using some eyeball model, before actual surgery of peeling of the inner limiting membrane.

Conventionally, training for skill acquisition in general ophthalmic surgery has been performed using enucleated animal eyes (typically from pigs). However, understanding of the unique structure of the human eyeball is essential in advanced eye surgery, and alternative training using enucleated animal eyes has become unsuitable for training in inner limiting membrane peeling. In this respect, for instance, Patent Literature 1 discloses an artificial eye model for ophthalmic surgery training, provided with a membrane that simulates the inner limiting membrane. Further, Patent Literature 2 discloses an artificial inner limiting membrane peeling model that can be used for the purpose of training in peeling of the inner limiting membrane.

### CITATION LIST

### Patent Literature

### Patent Literature 1: U.S. Patent Application Publication No. 2012/0021397

### Patent Literature 2: WO 2015/151939

### SUMMARY OF INVENTION

However, the artificial eyeball model disclosed in Patent Literature 1 is not a structure corresponding to training in surgery of peeling of the inner limiting membrane from the retina. The inner limiting membrane peeling model disclosed in Patent Literature 2 is ostensibly an inner limiting membrane peeling model that allows performing good procedure training, but the model itself is implemented for procedure training in a dry state, whereas a model would be preferable that enables procedure training in a wet state, which is a condition closer to that of an actual human eye.

Therefore, the present invention has been made with an object for providing an inner limiting membrane peeling model (material for procedure training), different from conventional models for procedure training, and which allows performing procedure training in peeling of the inner limiting membrane under wet conditions, similar to the natural state inside the human eyeball.

In order to achieve the above object, the present invention provides an inner limiting membrane peeling model for use in procedure training in peeling of an inner limiting membrane, the model including a simulated retina, and a simulated inner limiting membrane formed on the simulated retina.

In the inner limiting membrane peeling model disclosed herein, the simulated inner limiting membrane is formed of a hydrophilic polymer gel that is formed mainly of a water-soluble polymer (i.e. a component in excess of 50 mass % of the constituent components of the simulated inner limiting membrane; the same shall apply hereinafter); and the model is disposed in a state where the simulated inner limiting membrane is immersed in water or an aqueous solution containing a predetermined solute, at least during the above use (i.e. during procedure training in peeling of the inner limiting membrane).

The inner limiting membrane peeling model disclosed herein is a so-called wet-type inner limiting membrane peeling model in which, as described above, the simulated inner limiting membrane is formed of a hydrophilic polymer gel, and procedure training is performed in a state where the simulated inner limiting membrane is immersed in water or an aqueous solution containing a predetermined solute (hereinafter, these may also be referred collectively to as “aqueous medium”).

The terms “simulated retina” and “simulated inner limiting membrane” as used in the present invention respectively refer to a substrate for executing procedure training in peeling of the inner limiting membrane under wet conditions similar to those in the natural state inside the human eyeball, and to a membrane-like member that is peeled off that substrate, and are not necessarily identical, in material, shape or appearance, to an actual retina or an actual inner limiting membrane.

For instance, the simulated retina need not be an organic retina similar to that of an actual retina, and may be a substrate configured of an inorganic material of arbitrary shape, so long as the substrate can be used as a substrate for performing procedure training in peeling of the inner limiting membrane. Similarly, the simulated inner limiting membrane may be of any shape, so long as the membrane is suitable as membrane made of a hydrophilic polymer gel that is peeled from the substrate, in order to perform procedure training in peeling of the inner limiting membrane under wet conditions similar to the natural state inside the human eyeball.

The actual eyeball is filled with a liquid, so-called aqueous humor, that circulates in the eye; during ophthalmic surgery with peeling of the inner limiting membrane, an ocular reflux solution is caused to circulate, to adjust intraocular pressure, and for intraocular cleaning. Accordingly, performing procedure training with a model that simulates more faithfully an actual surgery environment is important herein in a surgery training system. Depending on the conventional technique (for instance, the inner limiting membrane peeling model disclosed in Patent Literature 2), however, it is difficult to perform procedure training in peeling of the inner limiting membrane in a wet environment that simulates actual ophthalmic surgery. By contrast, the wet-type inner limiting membrane peeling model disclosed herein allows performing training in peeling of the inner limiting membrane in a wet environment while under supply, to the simulated inner limiting membrane, of an aqueous solution that is actually used during surgery, such as water (for instance, distilled water or tap water), or an ocular reflux solution or saline. In consequence, this enables training in surgery of peeling of the inner limiting membrane, which demands a high level of skill, in an environment that approximates the wet environment at the time of actual ophthalmic surgery.

In a preferred mode of the wet-type inner limiting membrane peeling model disclosed herein, an average membrane thickness of the simulated inner limiting membrane is 0.5 μm to 20 μm.

By configuring the simulated inner limiting membrane out of a hydrophilic polymer gel having such membrane thickness, it becomes possible to achieve a thickness and feel similar to (typically identical to) those of a case where the inner limiting membrane of the human eye is gripped using a surgical instrument such as a forceps. Therefore, the feel of the inner limiting membrane of the human eye can be reproduced yet more suitably by prescribing the membrane thickness of the simulated inner limiting membrane to lie in the above range.

In another preferred mode of the wet-type inner limiting membrane peeling model disclosed herein, the hydrophilic polymer gel is configured mainly of at least one type selected from among a polyvinyl alcohol (PVA)-based resin, a polyethylene glycol (PEG)-based resin, collagen and gelatin.

Polymer gels configured of these polymer substances have good hydrophilicity (have water retention, in a yet more preferred case). In consequence, this enables good procedure training in peeling of the inner limiting membrane in an environment where an aqueous medium is used.

Particularly preferably, for instance, the hydrophilic polymer gel is configured mainly of a PVA-based resin having a saponification degree of 50% or higher and/or a PVA-based resin having a degree of polymerization of 300 to 3000, from the viewpoint of bringing the wet state of existence of the simulated inner limiting membrane closer to the natural state of existence of the inner limiting membrane in the human eye.

In another preferred mode of the wet-type inner limiting membrane peeling model disclosed herein, the simulated retina is mainly configured of a silicone-based resin (a component in excess of 50 mass % of the constituent components of the simulated retina; likewise hereinafter).

A simulated retina configured of a silicone-based resin makes it possible herein for the peelability of the simulated retina and adhesion thereof to the simulated inner limiting membrane, which is made of the hydrophilic polymer gel and is formed one surface of the simulated retina, to come close to the peelability and adhesion of the inner limiting membrane with a natural retina in an actual human eye, which in turns enables realistic training in peeling of the inner limiting membrane.

In another preferred mode of the wet-type inner limiting membrane peeling model disclosed herein, the simulated inner limiting membrane contains a coloring agent.

By imparting coloring to the simulated inner limiting membrane of the internal boundary peeling model, so that the membrane can be identified visually, it becomes possible to perform procedure training in peeling of the inner limiting membrane in an environment that is easier to grasp visually. Also, in actual inner limiting membrane peeling surgery in the human eye, the inner limiting membrane may in some instances be colored using a coloring agent, for the purpose of peeling the inner limiting membrane reliably and safely. Procedure training can be performed as a result in a state that conforms to actual surgery.

In a particularly preferred mode of the wet-type inner limiting membrane peeling model disclosed herein, the model is further provided with an outer wall portion formed to a human eyeball shape, such that the simulated retina and the simulated inner limiting membrane are disposed inward of the outer wall portion.

By being formed as an inner limiting membrane peeling model having a shape that approximates that of a human eye, the model allows performing procedure training with a feeling of high realism and immediacy.

Particularly preferred modes of the wet-type inner limiting membrane peeling model disclosed herein include those in (1) to (5) below.

(1). An inner limiting membrane peeling model for use in procedure training in peeling of the inner limiting membrane, the model including:

a simulated retina;

a simulated inner limiting membrane formed on the simulated retina, and disposed immersed in water or an aqueous solution containing a predetermined solute, at least during the above use; and

an outer wall portion having a shape resembling a human eyeball,

wherein the simulated retina and the simulated inner limiting membrane are disposed inward of the outer wall portion;

the simulated inner limiting membrane is formed of a hydrophilic polymer gel that is formed mainly of a water-soluble polymer; and

the hydrophilic polymer gel is mainly configured of a polyvinyl alcohol (PVA)-based resin having a saponification degree of 50% or higher, and a degree of polymerization of 300 to 3000.

(2). An inner limiting membrane peeling model for use in procedure training in peeling of the inner limiting membrane, the model including:

a simulated retina;

a simulated inner limiting membrane formed on the simulated retina; and

an outer wall portion having a shape resembling a human eyeball,

wherein the simulated retina and the simulated inner limiting membrane are disposed inward of the outer wall portion;

the simulated inner limiting membrane is disposed immersed in water or an aqueous solution containing a predetermined solute;

the simulated inner limiting membrane is formed of a hydrophilic polymer gel that is formed mainly of a water-soluble polymer; and

the hydrophilic polymer gel is mainly configured of a polyvinyl alcohol (PVA)-based resin having a saponification degree of 50% or higher and a degree of polymerization of 300 to 3000.

(3). The inner limiting membrane peeling model of (1) or (2), wherein an average membrane thickness of the simulated inner limiting membrane is 0.5 μm to 20 μm.

(4). The inner limiting membrane peeling model of any one of (1) to (3), wherein the simulated retina is mainly configured of a silicone-based resin.

(5). The inner limiting membrane peeling model of any one of (1) to (4), wherein the simulated inner limiting membrane contains a coloring agent.

In order to attain the above goal, the present invention can provide also a kit (combination of articles) for procedure training in peeling of the inner limiting membrane, the kit being used for procedure training in peeling of the inner limiting membrane, and the kit including:

an inner limiting membrane peeling model having any one of the configurations disclosed herein; and

water or an aqueous solution containing a predetermined solute (i.e. aqueous medium), used for the purpose of submerging the simulated inner limiting membrane, at least during use.

The present invention can also provide an inner limiting membrane peeling training device that is used in procedure training in peeling of the inner limiting membrane. The inner limiting membrane peeling training device disclosed herein includes:

an inner limiting membrane peeling model setting portion; and

an inner limiting membrane peeling model having any one of the configurations disclosed herein set (fitted) to the setting portion.

As pointed out above, training in inner limiting membrane peeling can be performed in a wet environment similar to that of the actual human eye, through the use of the wet-type inner limiting membrane peeling model disclosed herein. Accordingly, the inner limiting membrane peeling training device provided by the present invention enables training in surgery of peeling of the inner limiting membrane, from which a high level of skill is demanded, in an environment that approximates the wet environment at the time of actual ophthalmic surgery.

### BRIEF DESCRIPTION OF DRAWINGS

FIG. 1 is a cross-sectional diagram illustrating schematically a configuration example of an inner limiting membrane peeling model.

FIG. 2 is a cross-sectional diagram illustrating schematically another configuration example of an inner limiting membrane peeling model.

FIG. 3 is an explanatory diagram illustrating schematically an example of an inner limiting membrane peeling model formed in the shape of a human eye, and of an inner limiting membrane peeling training device to which the model can be fitted.

FIG. 4 is an explanatory diagram illustrating schematically the configuration of an inner limiting membrane peeling training device to which there is fitted an inner limiting membrane peeling model formed in the shape of a human eye.

### DESCRIPTION OF EMBODIMENTS

Preferred embodiments of the present invention will be explained below. Any features other than the matter specifically set forth in the present description and that may be necessary for carrying out the present invention can be regarded as design matter for a person skilled in the art based on conventional techniques in the relevant technical field. The present invention can be realized on the basis of the disclosure of the present description and common technical knowledge in the relevant technical field. In the drawings below, members and portions that elicit identical effects are denoted with identical reference symbols, and a recurrent explanation thereof will be omitted or simplified. The dimensional relationships (length, width, thickness and so forth) in the figures do not reflect actual dimensional relationships. In the present description, a numerical value range notated as “A to B” (where A and B are any numerical values) denotes a value equal to or larger than A and equal to or smaller than B.

The inner limiting membrane peeling model disclosed herein is a model provided with a simulated retina and with a simulated inner limiting membrane formed on the simulated retina. The shape of the inner limiting membrane peeling model disclosed herein is not particularly limited, so long as the shape enables procedure training in the targeted surgery of peeling of the inner limiting membrane. For instance, the shape may be sheet-like, plate-like, or chip-like, of appropriate size.

FIG. 1 illustrates schematically a typical example of the configuration of the inner limiting membrane peeling model disclosed herein. The inner limiting membrane peeling model 10 is provided with a sheet-shaped simulated retina 30 (i.e. substrate for forming a simulated inner limiting membrane 20), and with a membrane-shaped (sheet-shaped) simulated inner limiting membrane 20 that is laid up on one surface (one side) of the simulated retina. Such inner limiting membrane peeling model is used in preparatory procedure training for surgery of peeling of the inner limiting membrane in which the inner limiting membrane is peeled off the retina. Accordingly, prior to use (i.e. prior to procedure training in peeling of the inner limiting membrane; typically, during storage), the surface of the model can be protected with a protective sheet for preventing dry-out, without supply of an aqueous medium.

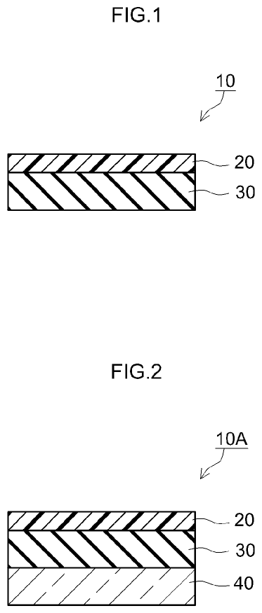

Alternatively, an inner limiting membrane peeling model 10A is preferred of a form where the model is provided with some support substrate 40 on the back side of the simulated retina 30, as illustrated in FIG. 2. The inner limiting membrane peeling model 10A implemented by being provided with such a support substrate 40 boasts superior shape retention, and therefore is preferred herein.

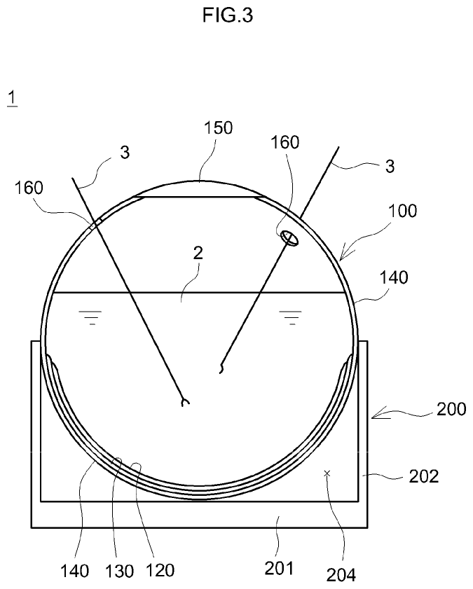

The surface shape of the simulated inner limiting membrane of the inner limiting membrane peeling model may be flat, such as that in FIG. 1 and FIG. 2, and may be curved mimicking the concave surface of the fundus sphere of the human eye, as illustrated in FIG. 3. Specifically, the support substrate in an inner limiting membrane peeling model 100 having a human eye shape (i.e. shape resembling a human eye), such as the one FIG. 3, corresponds to a simulated sclera 140 that constitutes an eyeball outer wall portion, with a simulated retina 130 and a simulated inner limiting membrane 120 are formed on the inward side of the simulated sclera 140.

The simulated sclera (eyeball outer wall portion) 140 is formed through molding, out of an appropriate synthetic resin material or elastomer material. In surgery of peeling of the inner limiting membrane on the human eye, it is often the case that the area of the inner limiting membrane to be peeled is small (for instance, about the size of a circle having a diameter of 3 mm to 5 mm). Accordingly, the present invention can be suitably realized both as the inner limiting membrane peeling model 100 having a curved shape that imitates the concave surface of the fundus sphere of the human eye, or as the internal boundary peeling models 10, 10A of planar shape.

The peelability of the inner limiting membrane peeling model disclosed herein during peeling of the simulated inner limiting membrane from the simulated retina is similar to the peelability during peeling of the natural inner limiting membrane from the retina in a human eye. The similarity of the peelability during peeling of the simulated inner limiting membrane from the simulated retina, with respect to the peelability during peeling of the inner limiting membrane in a human eye, can be evaluated herein by performing a sensory test in which a doctor (i.e. person skilled in the art), familiarized the procedure of inner limiting membrane peeling in a human eye, peels the simulated inner limiting membrane from the simulated retina. For instance, the similarity in peelability can be evaluated by resorting to the evaluation method illustrated in the examples described below. Alternatively, the similarity in peelability can be evaluated by measuring, for instance, the breaking strength, the elongation at break and/or the peeling strength of the inner limiting membrane peeling model.

The peelability (for instance, breaking strength and elongation at break) of such inner limiting membrane peeling model can be adjusted by adjusting as appropriate, for instance, the properties (membrane thickness and strength) of the hydrophilic polymer gel that constitutes the simulated inner limiting membrane, or by controlling as appropriate, for instance, the molecular weight, degree of polymerization, saponification degree, presence or absence and extent of chemical modification of functional groups in the main chain (polymer skeleton) and in side chains, of the water-soluble polymer material for forming the polymer gel. Alternatively, the peelability of the inner limiting membrane peeling model can be adjusted by adjusting as appropriate the properties (membrane thickness and strength) of the simulated retina, or by controlling as appropriate, for instance, the molecular weight, degree of polymerization, saponification degree, presence or absence and extent of chemical modification of functional groups in the main chain (polymer skeleton) and in side chains, of the polymer material that constitutes the simulated retina.

The thickness of the simulated inner limiting membrane in the wet-type inner limiting membrane peeling model disclosed herein is not particularly limited. The average membrane thickness of the simulated inner limiting membrane is appropriately 0.5 μm or greater (preferably 1 μm or greater, for instance 2 μm or greater, or 3 μm or greater), and is preferably 20 μm or smaller (preferably 15 μm or smaller, for instance 10 μm or smaller or 3 μm or smaller), from the viewpoint of reproducing, at a high level, a peelability that approximates the peelability of the inner limiting membrane peeling in a human eye (typically for instance the grippability, breaking strength and elongation at break of the simulated inner limiting membrane).

For instance, procedure training in peeling of the inner limiting membrane in wet environment can be carried out more suitably by setting the average membrane thickness of the simulated inner limiting membrane configured of a hydrophilic polymer gel to lie in the above ranges (for instance 0.5 μm to 20 μm, or 0.5 μm to 3 μm). In the present description, the terms “membrane thickness” and “thickness” respectively denote average membrane thickness and average thickness; preferably, however, the simulated inner limiting membrane lies, over 90% or more of the entire measurement region, within the ranges of membrane thickness or thickness set forth herein.

The simulated inner limiting membrane of the inner limiting membrane peeling model disclosed herein is configured of a hydrophilic polymer gel that is formed mainly of a water-soluble polymer, so as to enable training in peeling of the inner limiting membrane in a wet environment. Preferred examples of the water-soluble polymer include herein water-soluble polymers derived from proteins, for instance, various types of collagen (for example IV-type collagen and I-type atelocollagen), water-soluble proteins such as gelatin, as well as water-soluble polymers such as polyvinyl alcohol (PVA)-based resins and polyethylene glycol (PEG)-based resins.

The term “polyethylene glycol (PEG)-based resin” encompasses a polymer (polyether), the molecular chain (main chain) of which is configured of PEG units: −(CH₂—CH₂—O)ₙ—, the PEG being modified with various functional groups. Examples include polyethylene glycol diacrylate, polyethylene glycol dimethacrylate, alkoxy polyethylene glycol methacrylates and alkyl phenoxy polyethylene glycol acrylates.

The term “polyvinyl alcohol (PVA)-based resin” denotes herein not only PVA represented by

Formula: (—CH₂CH(OH)—)ₙ in a narrow sense (i.e. having a saponification degree of 100%), but also encompasses various polymers having a saponification degree of (l/(l+m)×100) and a degree of polymerization of (l+m), and represented by Formula: —(CH₂CH(OH))ₗ—(CH₂CH(OCOCH₃))ₘ—. Further, the term “polyvinyl alcohol (PVA)-based resin” encompasses materials (polymers) in which some hydroxyl groups or acetic acid groups in the above-mentioned polymer are substituted with other functional groups. For instance, it is preferable herein to use a PVA-based resin having a saponification degree of 78% or higher (for instance 80% or higher), and further 85% or higher, and in particular 90% or higher, since in that case good hydrophilicity can be brought out. A degree of polymerization of 300 or higher is suitable for use; preferably, the degree of polymerization is 1000 or higher, and particularly preferably 1700 or higher. The upper limit of the degree of polymerization may be about 3000. Good hydrophilicity (and water retention) and gel mechanical strength both can be achieved in a balanced manner when the degree of polymerization is 1000 or higher (in particular 1700 or higher) and 3000 or lower (in particular 2400 or lower). The molecular weight of a PVA-based resin having such preferred degree of polymerization can lie herein in the range of about 10000 to 150000.

A hydrophilic polymer gel (hydrogel) insoluble in water can be obtained through cross-linking of such a polymer compound using an appropriate cross-linking agent and a catalyst.

A hydrophilic polymer gel can be formed as a result of suitable crosslinking reaction by using, as the cross-linking agent, a general conventionally used compound, for instance glutaraldehyde, N,N′-methylenebisacrylamide or hexamethylenetetramine, and by using an inorganic acid or an organic acid (for instance, hydrochloric acid or acetic acid) as the catalyst. The crosslinking reactions involved and the use of cross-linking agents are mere instances of conventional art, and a further detailed explanation thereof will be omitted herein.

The hydrophilic polymer gel that constitutes the simulated inner limiting membrane can contain, for instance, a heat stabilizer, a plasticizer, a lubricant, an antioxidant, a filler, a surfactant, a stabilizer, a pH adjuster and/or a coloring agent (dye or pigment), as needed. Using a coloring agent is particularly preferred herein.

For instance, it is preferable to add, as a filler, a substance that can be colored, and to add a compound (coloring agent) capable of imparting color to that substance. The simulated inner limiting membrane may be colorless and transparent, but it is preferable that the simulated inner limiting membrane be colored by using a coloring, in order to peel the simulated inner limiting membrane reliably and safely. In the case, for instance, where the polymer gel is formed mainly of a PVA-based resin or a PEG-based resin, for instance, a protein-based substance such as gelatin may be added to a material for polymer gel preparation, and there may be further added a coloring agent (for instance, Brilliant Blue, Coomassie Brilliant Blue or a fluorescent dye) capable of staining the protein-based substance. Pigments (for instance, azo pigments, phthalocyanine pigments and anthraquinone pigments) can be used herein, without particular limitations, as the coloring agent.

The simulated retina will be explained next. The simulated retina disclosed herein is not particularly limited so long as it is a substrate that can bring about, together with the simulated inner limiting membrane, peelability (typically the above breaking strength, elongation at break and peeling strength) that approximates the peelability in inner limiting membrane peeling in a human eye. The thickness of the simulated retina (substrate) is not particularly limited and can be set as appropriate, for instance, from the viewpoint of productivity, cost and storability. For instance, the average membrane thickness of the simulated retina can be set to about 100 μm or more (for instance 200 μm or more) and 1000 μm or less (for instance 500 μm or less).

The material of the simulated retina is not particularly limited, and, for instance, the simulated retina may be formed mainly of an organic material such as an elastomer material or a synthetic resin material. Particularly preferably, the simulated retina is formed mainly of an elastomer material, since in that case peelability that approximates peelability at the time of peeling of the inner limiting membrane in an actual human eye can be easily realized at a high level. For instance, the simulated retina can be configured of a material having, as a main component, a polymer material such as a silicone rubber, butadiene rubber, isoprene rubber, butyl rubber, fluororubber, ethylene propylene rubber, nitrile rubber or natural rubber. Such a polymer material may be used singly, or as a combination of two or more types.

The simulated retina is formed particularly preferably of silicone rubber. The silicone rubber that is used is not particularly limited, so long as it is a polysiloxane having a crosslinked structure and rubber-like properties. Ordinarily, silicone rubbers are produced through cross-linking of a polysiloxane. The polysiloxane may be linear, branched or cyclic. Various functional groups may be introduced into the side chains of the polysiloxane being the main chain that constitutes the silicone rubber. Silicone rubber containing polydimethyl siloxane (PDMS; typically polydimethyl siloxane with both ends modified) in which substantially all side chains are methyl groups can be suitably used herein.

Besides the polysiloxane component that constitutes the silicone rubber, other known additives can be appropriately added to the simulated retina as needed, for instance a catalyst, a filler, an antioxidant, an ultraviolet absorber, a plasticizer, a coloring agent (dye or pigment), a reaction aid and/or a reaction inhibitor. For instance, the hardness of the silicone rubber can be adjusted as appropriate through adjustment of the addition amount of the catalyst, filler, plasticizer and so forth. As the silicone rubber there can be used a silicone rubber resulting from preparing or procuring as appropriate components such as those described above, or a commercial product that contains components such as those described above.

The support substrate will be explained next. The properties of the support substrate of the inner limiting membrane peeling model disclosed herein are not particularly limited, so long as the substrate can support the simulated inner limiting membrane and the simulated retina. The support substrate in the inner limiting membrane peeling model formed to a human eye shape can be herein a simulated sclera that constitutes the outer wall portion of the eyeball. The thickness of the support substrate is not particularly limited and can be set as appropriate, for instance, from the viewpoint of productivity, cost and storability. The average membrane thickness of the outer wall portion (simulated sclera) of the human eye model as the support substrate can be set to about 3 mm or less (preferably 0.5 mm to 2 mm, for instance 1 mm±0.2 mm). The inner limiting membrane peeling model disclosed herein having a shape that resembles the human eye may take a form where the outer wall portion of the human eye model is other than a simulated sclera, so long as the model can be suitably used for procedure training in surgery of peeling of the inner limiting membrane of the eyeball.

The material of the support substrate (for instance simulated sclera) is not particularly limited. For instance, the simulated sclera may be formed mainly of an organic material such as an elastomer material or a synthetic resin material. Particularly preferably, the simulated sclera is formed of mainly of an elastomer material, to approximate an actual human eye. For instance, the simulated sclera can be configured of a material having, as a main component, a polymer material such as a silicone rubber, butadiene rubber, isoprene rubber, butyl rubber, fluororubber, ethylene propylene rubber, nitrile rubber or natural rubber. Such a polymer material may be used singly, or as a combination of two or more types.

In particular, the support substrate (particularly the simulated sclera) is formed of a silicone rubber. The silicone rubber that is used is not particularly limited, so long as it is a polysiloxane having a crosslinked structure and rubber-like properties. The polysiloxane may be linear, branched or cyclic. Various functional groups may be introduced into the side chain of the polysiloxane being the main chain that constitutes the silicone rubber. Silicone rubber containing polydimethyl siloxane (PDMS; for instance polydimethyl siloxane with both ends modified) in which substantially all side chains are methyl groups can be suitably used herein.

Other additives, such as a catalyst, a filler, an antioxidant, an ultraviolet absorber, a plasticizer, a coloring agent (dye or pigment), a reaction aid and/or a reaction inhibitor, can be appropriately added to the support substrate as needed. For instance, the hardness of the silicone rubber can be adjusted as appropriate through adjustment of the addition amount of the catalyst, filler, plasticizer and so forth. As the silicone rubber there can be used a silicone rubber resulting from preparing or procuring, as appropriate, components such as those described above, or a commercial product that contains components such as those described above. This feature itself is an instance of conventional art and a particularly detailed explanation thereof will be omitted herein.

Production (formation) of the inner limiting membrane peeling model disclosed herein will be explained next.

The constituent elements themselves that make up the inner limiting membrane peeling model disclosed herein are constructed out of conventionally known materials, as pointed out above (for instance, the hydrophilic polymer gel configured of a PVA-based resin, silicone rubber and so forth). The inner limiting membrane peeling model can be produced (formed) by resorting to a conventional formation method that matches the material.

In the case, for instance, where various kinds of elastomer material or synthetic resin material is used as a main constituent, the simulated retina can be formed by directly applying (typically directly coating) a liquid composition for forming a simulated retina onto a support substrate, followed as needed by drying and by performing a curing treatment of various types, such as photocuring or thermal curing. Specifically, the composition for forming a simulated retina can be coated onto the support substrate using, for instance, a gravure coater, a roll coater, a die coater, a spin coater or a spray coater. Coating using a spin coater (i.e. spin coating method) is particularly preferred in order to carry out the present invention, since spin coating is excellent in workability and allows forming a thin membrane having uniform membrane thickness, with high precision.

Alternatively, a material containing the above material as a main component may be used, for forming the membrane in accordance with a general membrane (sheet) molding method (for instance extrusion or an inflation molding method). Such methods are suitable for forming a simulated retina in an inner limiting membrane peeling model that lacks a support substrate. A simulated retina formed relying on such a method may be utilized while fixed to the support substrate, using, for instance, an adhesive.

A conventionally known method can be selected, without particular limitations, as the method for providing the simulated inner limiting membrane on the simulated retina, so long as the method allows producing a simulated inner limiting membrane having peelability that approximates the peelability in inner limiting membrane peeling in a human eye.

For instance, the method may involve directly coating the simulated retina, in accordance various coating methods (for instance spin coating), with a liquid or slurry-like composition for forming a simulated inner limiting membrane containing a constituent component (hydrophilic polymer gel) of the simulated inner limiting membrane, with heating or irradiation, depending on the type of cross-linking agent, to elicit cross-linking of the coated product, and produce thereby the intended polymer gel.

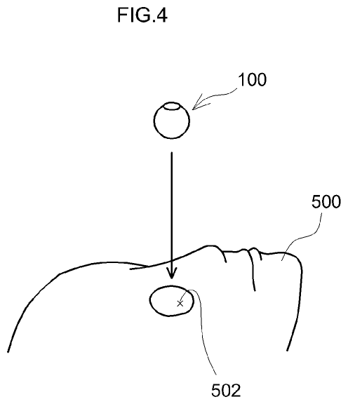

One embodiment of a suitable human eye model, and of an inner limiting membrane peeling training device provided with the human eye model will be explained next, with reference to FIG. 3 and FIG. 4, as the inner limiting membrane peeling model disclosed herein.

As illustrated in FIG. 3, an inner limiting membrane peeling training device 1 according to the present embodiment is configured of: an inner limiting membrane peeling model 100 formed to a shape and size resembling those of a human eye, and a setting portion 200 of the inner limiting membrane peeling model (hereinafter simply referred to as “setting portion 200”) for setting (fitting) the inner limiting membrane peeling model 100.

As illustrated in the figures, specifically, the inner limiting membrane peeling model 100 according to the present embodiment is formed to a hollow spherical shape having a diameter of about 24 mm, close to that of a human eye. The outer wall portion (spherical surface), which is a molded product (support substrate) configured of silicone rubber and has a thickness of about 1 mm±0.1 mm, constitutes a simulated sclera 140 that is formed to a shape and size resembling those of a human eye. Although not particularly limited thereto, the modulus of elasticity (based on a tensile test performed on a membrane (sheet) according to JIS or ASTM; likewise hereinafter) of the simulated sclera 140 configured of silicone rubber is adjusted to be about 0.5 MPa to 20 MPa, so as to approximate that of the human eye. Preferably, the modulus of elasticity is about 1 MPa to 10 MPa.

A portion of the outer wall corresponding to the front of the human eye constitutes a slightly raised corneal region 150, similarly to that of the human eye. As a result, the positional relationship with respect to the below-described setting portion 200 can be properly secured.

As illustrated in FIG. 3, the simulated retina 130 is formed at a region opposing the corneal region 150 on the inward side of the simulated sclera (outer wall portion) 140 of the inner limiting membrane peeling model 100, specifically at a region in which there are present the optic nerve and the macular area in a natural human eye, for instance, at a region corresponding to ⅓ to ½ of the entire interior of the eyeball, centered on the macular area of the human eye. The simulated retina 130 according to the present embodiment is formed of a silicone rubber, and has an average membrane thickness in the range of 100 μm to 500 μm (for instance 200 μm to 300 μm). Preferably, the modulus of elasticity of the simulated retina 130 configured of the silicone rubber is adjusted to be smaller than about 100 kPa (for instance to be 10 kPa to 50 kPa), so as to approximate that of the human eye.

The simulated inner limiting membrane 120 according to the present embodiment is formed on the top face of the simulated retina 130. Specifically, the simulated inner limiting membrane 120 is formed by a hydrophilic polymer gel resulting from cross-linking of a PVA-based resin having a saponification degree of 50% or higher and a degree of polymerization of 300 to 3000 (more preferably a PVA-based resin having a saponification degree of 78% or higher and/or a degree of polymerization from 1000 to 3000). The average membrane thickness of the simulated inner limiting membrane 120 is 3 μm to 15 μm. Alternatively, the average membrane thickness of the simulated inner limiting membrane 120 may be set to 1 μm to 3 μm, so as to approximate a simulated inner limiting membrane of a human living eyeball. The modulus of elasticity of the simulated inner limiting membrane 120 configured of the hydrophilic polymer gel is adjusted to about 50 kPa to 200 kPa (for instance 100 kPa to 150 kPa), so as to approximate that of the human eye.

As illustrated in FIG. 3, the setting portion 200 according to the present embodiment is configured of a base plate 201, and a cylindrical peripheral wall 202 rising from the base plate and having an inner diameter that corresponds to the diameter of the inner limiting membrane peeling model 100 of human eye shape.

In such a configuration, the inner limiting membrane peeling model 100 having the above human eye shape is fitted to the setting portion 200, in a state where about half the inner limiting membrane peeling model 100 is accommodated in a mounting space 204 that is surrounded by the peripheral wall 202 of the setting portion 200, as illustrated in FIG. 3. As a result, procedure training in peeling of the inner limiting membrane can be carried out in a physically stable form.

As illustrated in FIG. 4, there can be used an inner limiting membrane peeling training device, prepared beforehand, having a face model 500 that is formed to resemble a human face. In this mode, a fitting hole (i.e. a hole constituting a simulated orbit) 502 corresponds to a concave setting portion 502 into which there is fitted the inner limiting membrane peeling model 100 of human eye shape. Procedure training in peeling of the inner limiting membrane can be performed by fitting the inner limiting membrane peeling model 100 to the setting portion (simulated orbit) 502. This mode allows procedure training in peeling of the inner limiting membrane to be carried out more realistically.

To use the inner limiting membrane peeling training device 1 illustrated in FIG. 3 and FIG. 4, specifically an aqueous medium 2 (water or an aqueous solution containing a predetermined solute, for instance, distilled water of saline) provided in, for instance, the kit disclosed herein, is supplied into the inner limiting membrane peeling model 100 of human eye shape, prior to procedure training in peeling of the inner limiting membrane, to form an environment in which at least the inner limiting membrane 120 is immersed in the supplied aqueous medium 2. As a result, procedure training in peeling of the inner limiting membrane can be carried out in a physically stable form, preferably in a wet environment. The inner limiting membrane peeling model 100 of human eye shape can be produced and sold in a state where the interior of the simulated sclera (outer wall portion) 140 contains beforehand the aqueous medium 2, and the simulated inner limiting membrane 120 is immersed in water or an aqueous solution containing a predetermined solute. This implementation allows dispensing with the trouble of supplying the aqueous medium 2 into the inner limiting membrane peeling model 100 immediately prior to procedure training in peeling of the inner limiting membrane. The supply amount of the aqueous medium 2 is not particularly limited, so long as an environment can be formed in which the inner limiting membrane 120 is immersed in the supplied aqueous medium 2. For instance, the supply amount of the aqueous medium 2 may be increased up to reaching the vicinity of the corneal region 150 illustrated in FIG. 3.

An insertion port 160 is provided in part of the simulated sclera 140, and the user can perform procedure training in peeling of the inner limiting membrane by inserting an appropriate surgical instrument 3 (forceps, cannula or the like) into the eyeball (simulated sclera 140), through the insertion port 160. The insertion port 160 may be formed beforehand, or may be formed directly by the user, by manipulating, for instance, the surgical instrument at the time of procedure training.

Several examples of the present invention will be explained next, but the invention is not meant to be limited to such examples.

### Test Example 1: Production of an Inner Limiting Membrane Peeling Model

A total of 11 types (sample Nos. 1 to 11) of inner limiting membranes given in Table 1 were produced using respective PVA-based resins having mutually different degrees of polymerization and saponification degrees, to thereby produce a total of 11 types (sample Nos. 1 to 11) of inner limiting membrane peeling models having mutually different inner limiting membranes.

TABLE 1

| Sample No. | Degree of polymerization | Saponification degree |
|---|---|---|
| 1 | 300 | 98% |
| 2 | 300 | 88% |
| 3 | 300 | 78% |
| 4 | 1000 | 98% |
| 5 | 1000 | 88% |
| 6 | 1700 | 98% |
| 7 | 1700 | 88% |
| 8 | 1700 | 78% |
| 9 | 2400 | 98% |
| 10 | 2400 | 88% |
| 11 | 2400 | 78% |

A 0.25 mm-thick glass plate was prepared as a support substrate. A silicone rubber coating material resulting from mixing a heat-curable dimethyl silicone rubber (product name “DOW CORNING TORAY SILPOT 184 W/C” by Dow Corning Toray Co., Ltd.), as a silicone rubber material having polydimethyl siloxane as a main component, with 1 part by mass of a curing catalyst with respect to 10 parts by mass of the main constituent, was applied onto one face of the support substrate by spin coating (rotational speed 1000 rpm, rotation time 30 seconds). The glass plate having been coated with the silicone rubber coating material was heated for about 10 minutes on a hot plate at 90° C., to dry and cure the silicone rubber, and produce thereby a circular simulated retina having a diameter of about 10 mm. The average thickness of the simulated retina was about 280 μm.

An inner limiting membrane was formed subsequently on the thus-produced simulated retina. This was accomplished as follows.

(1) Glutaraldehyde as a cross-linking agent, Brilliant Blue as a coloring agent and gelatin as a filler for coloring were prepared, using any one of the PVA-based resins given in Table 1.

(2) Respective aqueous materials for inner limiting membrane formation were then prepared by adding each material described in (1) above to distilled water, with mixing, to yield concentrations of PVA-based resin: 100 mM, cross-linking agent: 500 mM, filler for coloring: 1 mM, and coloring agent: 40 μM.

(3) The simulated retina produced above was coated with each aqueous material for inner limiting membrane formation, and was dried in a chamber. Thereafter, the whole was subjected to a heating treatment at 70° C. for 1 hour, and further at 120° C. for 1 hour.

(4) Each sample after heating was next immersed for 2 to 10 minutes in a 1 M solution of hydrochloric acid (HCl) as a catalyst, at room temperature, to elicit a crosslinking reaction.

(5) A respective simulated inner limiting membrane configured of a hydrophilic polymer gel (hydrogel) resulting from cross-linking of a PVA-based resin by glutaraldehyde became thus formed on the simulated retina. The average thickness of all the simulated inner limiting membranes lay in the range of 1 to 6 μm.

### Test Example 2: Evaluation of the Peelability of the Inner Limiting Membrane Peeling Model

An inner limiting membrane peeling test in accordance with the method below was performed on the inner limiting membrane peeling models of Examples 1 to 11, produced in Test example 1.

Test performers 1 and 2, who were herein two doctors (ophthalmologists) as persons skilled in the art, performed a procedure test of peeling of the simulated inner limiting membrane from the simulated retina, for each inner limiting membrane peeling model of the examples, using forceps utilized in actual surgery. Distilled water was supplied to each inner limiting membrane peeling model, and a peeling test was performed with the model immersed in distilled water up to the top face of the simulated inner limiting membrane.

The two doctors as the test performers conducted the test randomly on the inner limiting membrane peeling models of Examples 1 to 11, without knowing which sample each given specimen was. Test performers 1 and 2 are doctors having mastered, to a high level, the procedure of inner limiting membrane peeling.

The above peeling test was evaluated on the basis of the following criteria.

⊗: very close to the peelability at the time of actual peeling of the inner limiting membrane of a human eye

◯: similar to the peelability at the time of actual peeling of the inner limiting membrane of a human eye

Δ: perceivable feeling of strangeness as compared with the peelability at the time of actual peeling of the inner limiting membrane of a human eye

Table 2 sets out the evaluation (sensory test results) of peelability of the inner limiting membrane peeling model of each example, conducted by the test performers.

TABLE 2

| Sample No. | Test performer 1 | Test performer 2 |
|---|---|---|
| 1 | Δ | Δ |
| 2 | ⊗ | Δ |
| 3 | ◯ | ◯ |
| 4 | ⊗ | ◯ |
| 5 | ⊗ | ⊗ |
| 6 | ◯ | ◯ |
| 7 | ⊗ | ◯ |
| 8 | ◯ | ◯ |
| 9 | ⊗ | ◯ |
| 10 | ⊗ | ◯ |
| 11 | ◯ | ◯ |

As Table 2 reveals, peeling could be completed successfully in all inner limiting membrane peeling models. It was thus found that the models can be used as inner limiting membrane peeling models.

In particular, the results of Table 2 revealed that a simulated inner limiting membrane configured of a hydrophilic polymer gel derived from a PVC-based resin having a saponification degree higher than 78%, specifically a saponification degree of 88% or higher, and a degree of polymerization of 1000 or higher (herein 1000 to 2400), is suitable for training in surgery of peeling of the inner limiting membrane in a wet environment.

### INDUSTRIAL APPLICABILITY

As described above, the present invention is an inner limiting membrane peeling model (material for procedure training) that allows performing procedure training in peeling of the inner limiting membrane under wet conditions, similar to the natural state within the human eye. As a result, this enables training in surgery of peeling of the inner limiting membrane, which demands a high level of skill, in an environment that approximates the wet environment at the time of actual ophthalmic surgery.

1. An inner limiting membrane peeling model for use in procedure training in peeling of an inner limiting membrane, the model comprising:
a simulated retina;
a simulated inner limiting membrane formed on the simulated retina, and disposed immersed in water or an aqueous solution containing a predetermined solute, at least during the use; and
an outer wall portion having a shape resembling a human eyeball,
wherein the simulated retina and the simulated inner limiting membrane are disposed inward of the outer wall portion;
the simulated inner limiting membrane is formed of a hydrophilic polymer gel that is formed mainly of a water-soluble polymer; and
the hydrophilic polymer gel is mainly configured of a polyvinyl alcohol (PVA)-based resin having a saponification degree of 50% or higher and a degree of polymerization of 300 to 3000.

2. The inner limiting membrane peeling model of claim 1, wherein the simulated inner limiting membrane is disposed immersed in water or an aqueous solution containing a predetermined solute.

3. The inner limiting membrane peeling model of claim 1, wherein an average membrane thickness of the simulated inner limiting membrane is 0.5 μm to 20 μm.

4. The inner limiting membrane peeling model of claim 1, wherein the simulated retina is mainly configured of a silicone-based resin.

5. The inner limiting membrane peeling model of claim 1, wherein the simulated inner limiting membrane contains a coloring agent.

6. A kit for procedure training in peeling of the inner limiting membrane, used for performing procedure training in peeling of the inner limiting membrane, the kit comprising:
the inner limiting membrane peeling model of claim 1; and
water or an aqueous solution containing a predetermined solute, used for the purpose of immersion of the simulated inner limiting membrane.

7. An inner limiting membrane peeling training device used for procedure training in peeling of the inner limiting membrane, the device comprising:
an inner limiting membrane peeling model setting portion; and
the inner limiting membrane peeling model of claim 1 to be set in the setting portion.

8. The inner limiting membrane peeling model of claim 2, wherein an average membrane thickness of the simulated inner limiting membrane is 0.5 μm to 20 μm.

9. The inner limiting membrane peeling model of claim 2, wherein the simulated retina is mainly configured of a silicone-based resin.

10. The inner limiting membrane peeling model of claim 2, wherein the simulated inner limiting membrane contains a coloring agent.
